(19) United States
(12) Patent Application Publication
(10) Pub. No.: US 2025/0270150 A1
(43) Pub. Date: Aug. 28, 2025

(54) SYSTEM FOR PRODUCING ENERGY AND BIOMETHANE FROM WASTE

(71) Applicant: NUOVO PIGNONE TECNOLOGIE S.r.l., Florence (IT)

(72) Inventors: Simone Amidei, Florence (IT); Ali Mansouri, Ludwigshafen am Rhein (DE); Yeidy Sorani Montenegro Camacho, Florence (IT)

(21) Appl. No.: 18/584,398

(22) Filed: Feb. 22, 2024

(51) Int. Cl. C07C 1/12; F01D 15/10; H02K 7/18

(52) U.S. Cl. CPC C07C 1/12; F01D 15/10; H02K 7/1823; F05D 2220/76

(57) ABSTRACT

A system for producing energy and methane includes a waste-to-energy unit configured to produce energy and a flue gas by combusting waste and an oxidizing agent having oxygen and a carbon dioxide (CO₂) separation unit configured to separate CO₂ from the flue gas to provide separated CO₂. The system also includes a bio-methanation unit configured to generate methane (CH₄), heat, and water using the separated CO₂ received from the CO₂ separation unit and received hydrogen (H₂) gas. The system further includes an electrolyzer coupled to a source of water (H₂O) and an electric power source supplying electricity and configured to split the H₂O to generate the oxygen used in the oxidizing agent and the H₂ gas used in the bio-methanation unit.

### BACKGROUND

Waste-to-Energy (WtE) units combust waste, typically municipal solid waste, to generate electricity and/or heat and thus reduce an amount of waste that needs to be disposed. Unfortunately, WtE units emit significant amounts of carbon dioxide from the combustion process. On the other hand, green energy solutions involving renewable energy sources are limited and expensive. Hence, alternative solutions would be welcomed for the transition from fossil fuels to renewable energy sources.

### BRIEF SUMMARY

An embodiment of a system for producing energy and methane, the system including a waste-to-energy (WtE) unit coupled to a supply of waste and configured to combust the waste using an input of an oxidizing agent having oxygen to produce energy and a flue gas, the WtE unit having an oxidizing agent input, a flue gas discharge output, and an energy discharge output; a carbon dioxide (CO₂) separation unit coupled to the flue gas discharge output of the WtE unit and configured to separate CO₂ from the flue gas to provide separated CO₂, the CO₂ separation unit having a separated CO₂ discharge output; a bio-methanation unit coupled to the separated CO₂ discharge output of the CO₂ separation unit and configured to generate methane (CH₄), heat, and water using the separated CO₂ received from the CO₂ separation unit and received hydrogen (H₂) gas, the bio-methanation unit having a H₂ gas input, a CO₂ gas input, a methane output, a heat output configured, and a water output; and an electrolyzer coupled to a source of water (H₂O) and an electric power source supplying electricity and configured to split the H₂O to generate the oxygen used in the oxidizing agent and the H₂ gas used in the bio-methanation unit, the electrolyzer having a water input, an electric power input, a H₂ gas output coupled to the H₂ gas input of the bio-methanation unit, and an oxidizing agent output coupled to the oxidizing agent input of the WtE unit.

An embodiment of a method for producing energy and methane, the method includes combusting a waste using an input of an oxidizing agent that includes oxygen from an electrolyzer to produce a flue gas and energy, the combustion being performed by a waste-to-energy (WtE) unit; separating CO₂ from the flue gas to provide separated CO₂, the separation being performed by a CO₂ separation unit; generating methane (CH₄), heat, and water using the separated CO₂ and received hydrogen (H₂) gas, the generation being performed by a bio-methanation unit; and splitting water (H₂O) received from a source of H₂O using electric power received from a renewable electric power source to generate the oxygen used in the combustion and the H₂ gas received by the bio-methanation unit, the splitting being performed by the electrolyzer.

### BRIEF DESCRIPTION OF THE DRAWINGS

The following descriptions should not be considered limiting in any way. With reference to the accompanying drawings, like elements are numbered alike:

FIG. 1 illustrates a simplified flow diagram of a system for producing energy and biomethane from waste coupled to a user facility;

FIG. 2 depicts aspects of one embodiment of a waste-to-energy unit;

FIGS. 3A-3C, collectively referred to as FIG. 3, depict aspects of various embodiments of a carbon dioxide separation unit;

FIG. 4 depicts aspects of a bio-methanation unit;

FIG. 5 depicts aspects of an electrolyzer;

FIG. 6 depicts aspects of a first embodiment of the WtE unit coupled to the user facility;

FIG. 7 depicts aspects of a second embodiment of the WtE unit coupled to the user facility;

FIG. 8 depicts aspects of a third embodiment of the WtE unit coupled to the user facility;

FIG. 9 depicts aspects of a fourth embodiment of the WtE unit coupled to the user facility;

FIG. 10 depicts aspects of a fifth embodiment of the WtE unit coupled to the user facility;

FIG. 11 depicts aspects of a sixth embodiment of the WtE unit coupled to the user facility; and

FIG. 12 is a flow chart for a method for producing energy and methane.

### DETAILED DESCRIPTION

A detailed description of one or more embodiments of the disclosed apparatus and method presented herein by way of exemplification and not limitation with reference to the figures, in which like elements are numbered alike.

In the figures, arrows representing conveyance of a fluid or a flowable or conveyable solid may include representing pipes or structures for directing a flow or conveyance. These arrows may also represent any associated components such as valves, pumps, mechanical connectors and fittings and the like needed for flowing and/or conveying the fluid or conveyable solid. Similarly, arrows used to represent conveyance of electric power may represent conductors, cables, electrical connectors, transformers, switchgear and the like needed for the conveyance. While not explicitly discussed or illustrated, the various components of the disclosed apparatus requiring power inherently include a power supply or connection to a power source. Locations where arrows leave or enter a component can represent output ports (or connectors) or input ports (or connectors), respectively, for fluid flow or connections for electrical components. Components may include remotely controlled actuators for controlling the components using a controller. The controller may receive information from sensors distributed throughout the disclosed apparatus for monitoring operation and providing feedback control when necessary. Arrows depicting heat transfer may inherently represent a working fluid that transfers the heat.

Disclosed are apparatuses and methods for generating methane (CH₄) and using carbon dioxide (CO₂) emitted from a Waste-to-Energy (WtE) unit, which also emits energy. The CO₂ is separated or concentrated from a flue gas emission of the WtE unit and provided to a bio-methanation unit that uses the CO₂ to generate the methane. The bio-methanation unit also uses hydrogen (H₂) gas generated by an electrolyzer in the electrolysis process. The electrolyzer uses green or renewable electric power to generate the H₂ gas and oxygen (O₂) gas, which is fed to the WtE unit for the combustion of waste. The O₂ may be combined with an oxidizer input. In one or more embodiments, air may be combined with the O₂ received from the electrolyzer for the combustion process.

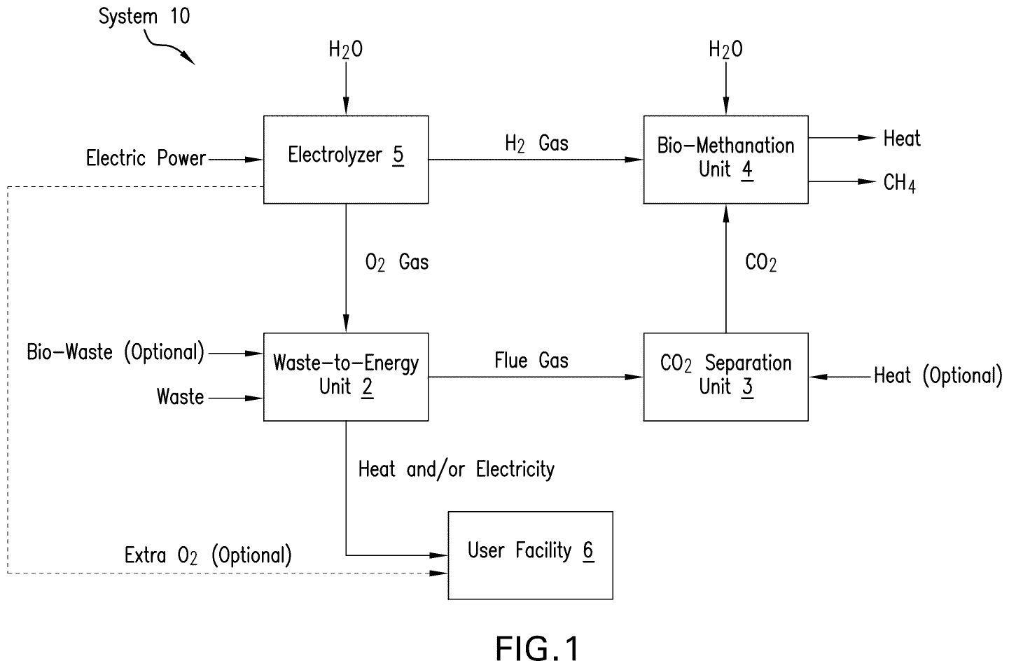

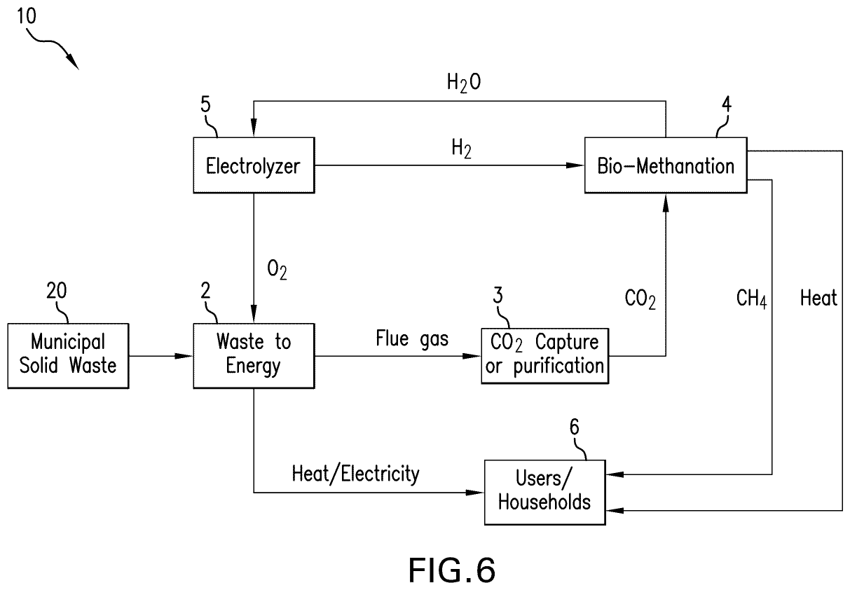

FIG. 1 illustrates a simplified flow diagram of a system 10 coupled to a user facility 6. The system 10 includes a WtE unit 2, a CO₂ separation unit 3, a bio-methanation unit 4, and an electrolyzer 5. The WtE unit 2 is any WtE unit that combusts a waste, such as solid waste obtained from a municipality, and generates electricity and/or heat that is provided to the user facility 6. Non-limiting examples of the waste include solid waste, liquid waste, organic waste, sewage waste, and/or a slurry of solid and liquid type wastes. The user facility 6 can be a commercial facility, an industrial facility, or a residential facility such as a house or apartment building in non-limiting embodiments. The WtE unit 2 has a flue gas discharge that is coupled to an input port of the CO₂ separation unit 3. The flue gas is a byproduct of the combustion of the waste, which uses oxygen received from the electrolyzer 5. The CO₂ separation unit 3 separates or concentrates the CO₂ in the received flue gas and provides the separated CO₂ through an output port to the bio-methanation unit 4. The term “separation unit” is intended to be inclusive of a CO₂ capture unit that captures CO₂ from the flue and then releases the captured CO₂ to the bio-methanation unit 4 thus separating and concentrating the CO₂ from the flue gas. Accordingly, the CO₂ separation unit 3 may use separation and/or capture processes to separate and concentrate the CO₂ in the flue gas. The CO₂ separation unit 3 may also optionally use heat in the CO₂ separation process received through a heat input port. The bio-methanation unit 4 generates methane using a bio-methanation process that uses the received separated CO₂ and hydrogen gas received from the electrolyzer 5. The bio-methanation process also generates heat and water (H₂O) that may be provided to other components through associated discharge ports. The electrolyzer 5 splits water molecules received as an input using electricity to generate the hydrogen gas provided to the bio-methanation unit 4 through a H₂ discharge port and an oxidant such as oxygen to the WtE unit 2 through an oxidant discharge port. In an embodiment where the electrolyzer 5 generates more oxygen than that needed in the combustion process in the WtE unit 2, the extra oxygen may be provided to the user facility 6 in various manners such as by flow via a tubular. The electricity for the electrolysis comes from a green or renewable energy source such as hydropower, wind turbines, and/or solar cells in non-limiting embodiments. FIGS. 6-11 discussed further below illustrate various embodiments of the system 10 coupled to a user facility 6.

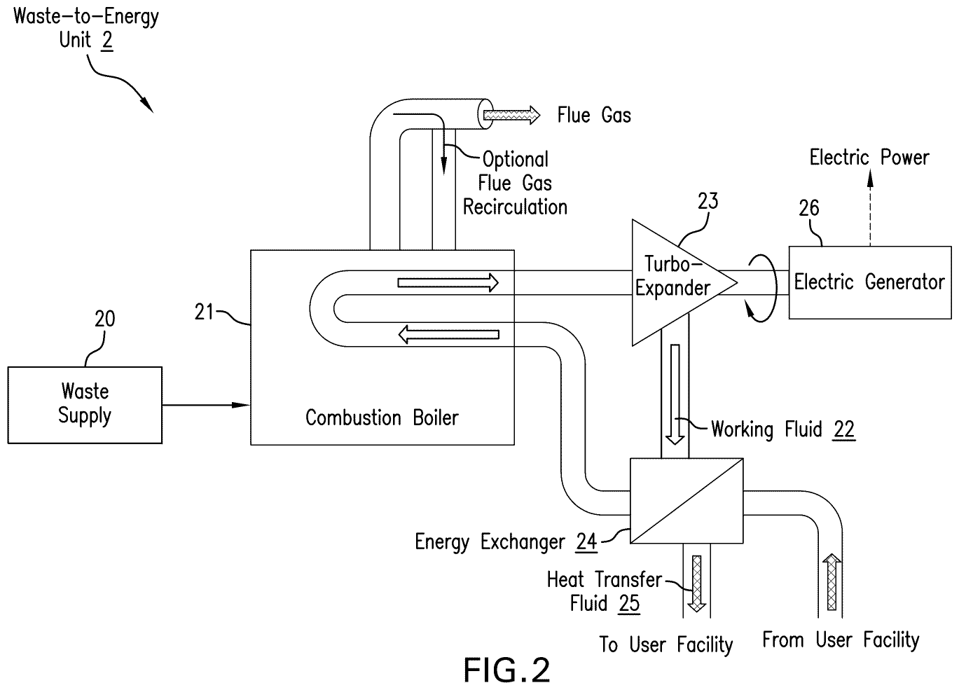

The WtE unit 2 may implement any one of several thermodynamic work cycles known in the art that produce heat and/or electricity. FIG. 2 illustrates one embodiment of the WtE unit 2. In the embodiment of FIG. 2, the WtE unit 2 implements a Brayton Cycle. A combustion boiler 21 (e.g., a furnace) combusts waste received from a waste supply 20. Optionally, the combustion boiler 21 may also combust bio-waste. The combustion heats a working fluid 22, such as water for example, in tubes in the combustion boiler 21. In embodiments where the WtE unit 2 implements an Allam Cycle, the working fluid 22 may be carbon dioxide. A flue gas having CO₂ is a byproduct of the combustion process and is discharged through a flue gas discharge port that is coupled to the CO₂ separation unit 3. Optionally, the flue gas may be recirculated through the combustion boiler 21 to produce the flue gas with mainly CO₂ and water vapor.

Still referring to FIG. 2, the heated working fluid 22 flows through and expands in a turbo-expander 23 that rotates a drive shaft coupled to an electric generator 26. The electric generator 26 generates electricity that can be provided to the user facility 6 or to other components of the system 10. An energy exchanger 24 downstream of the turbo-expander 23 provides a heat sink for the working fluid 22 that condenses the working fluid 22 to establish a pressure differential across the turbo-expander 23 causing the heated working fluid 22 to expand within the turbo-expander 23, thus causing the rotation of the drive shaft. In one or more embodiments, the energy exchanger 24 is a heat exchanger having a primary side (e.g., a shell-side) in fluid communication with the working fluid 22 and a secondary side (e.g., a tube side) in fluid communication with a heat transfer fluid 26. Accordingly, the heat transfer fluid 26 is heated using energy from the working fluid 22. The heat transfer fluid 26 can thus be used to provide energy to the user facility 6. Alternatively in one or more embodiments, the energy of the working fluid 22 can be provided directly to the user facility 6 by flowing the working fluid 22 directly to the user facility in a configuration referred to as “mass flow”. In that WtE plants that combust waste with a resultant flue gas to generate heat and/or electricity are known in the art, the WtE unit 2 and the various embodiments of the WtE unit 2 are not discussed in further detail.

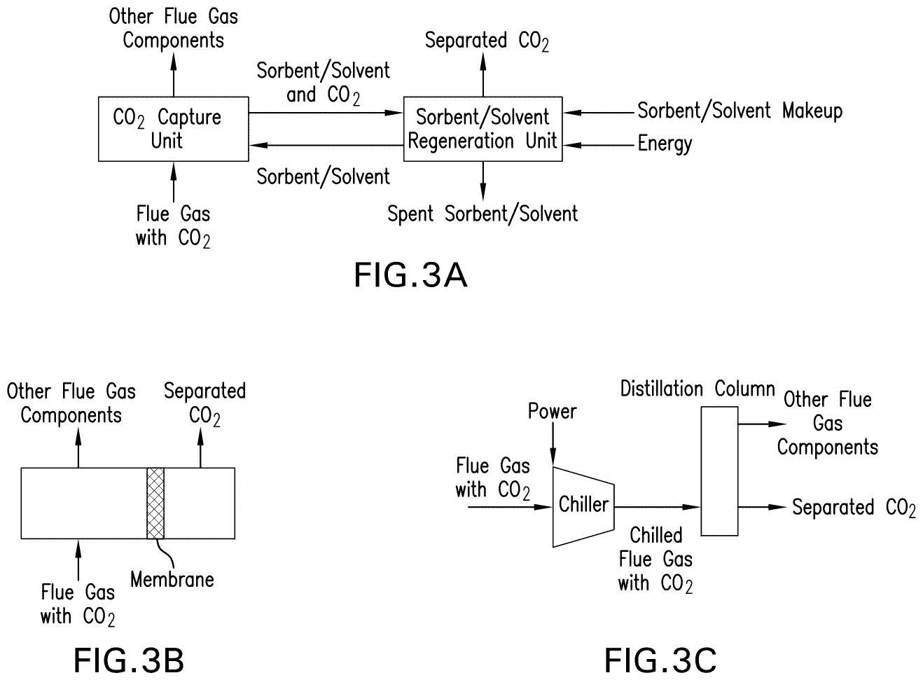

FIG. 3 illustrates three embodiments of processes implemented by the CO₂ separation unit 3. FIG. 3A illustrates a first process that separates CO₂ using chemical separation technology. In this process, a sorbent or solvent is mixed with the flue gas by flowing the flue gas through a packed bed of adsorbent at elevated pressure until the concentration of the gas mixture approaches equilibrium. Then, the combination of the CO₂ and the sorbent or solvent undergoes a sorbent/solvent regeneration process to release the CO₂ using pressure swing adsorption (PSA) or temperature swing adsorption (TSA). In PSA the bed is regenerated by reducing the pressure. In TSA the adsorbent is regenerated by raising its temperature. In one or more embodiments, amine (a solvent) scrubbing technology is used with mono-ethanolamine as a non-limiting example of a type of amine. Structurally, the flue gas is mixed with the sorbent or solvent in a mixer unit and then the CO₂ is released from the sorbent or solvent in a regeneration unit.

FIG. 3B illustrates a second process that separates CO₂ using membrane technology. CO₂ separation membranes allow the CO₂ in the flue gas to pass through faster than the other components. Non-limiting examples of a separation membrane include porous inorganic membranes, polymeric membranes, and zeolites. Multiple stages of membranes may be used to achieve a desired level of separation.

FIG. 3C illustrates a third process that separates CO₂ using cryogenic distillation. In this process, CO₂ is separated from other gases in the flue gas by cooling and condensation. Structurally, the flue gas is cooled in a chiller unit and then the CO₂ is separated in a distillation column.

In addition to the embodiments of FIGS. 3A-3C, condensation can also be used for separation in the case where the flue gas has a high concentration of CO₂. Additionally, a chilled ammonia process may also be used.

Structurally with respect to FIGS. 3A-3C, the CO₂ separation unit 3 includes an input port for receiving the flue gas from the WtE unit 2, an active separation element, and an output port for discharging the separated CO₂. The active separation element may be implemented as at least one of a chemical such as a sorbent or solvent, a membrane, or a distillation column. In that various CO₂ separation units are known in the art, the CO₂ separation unit 3 is not discussed in further detail herein.

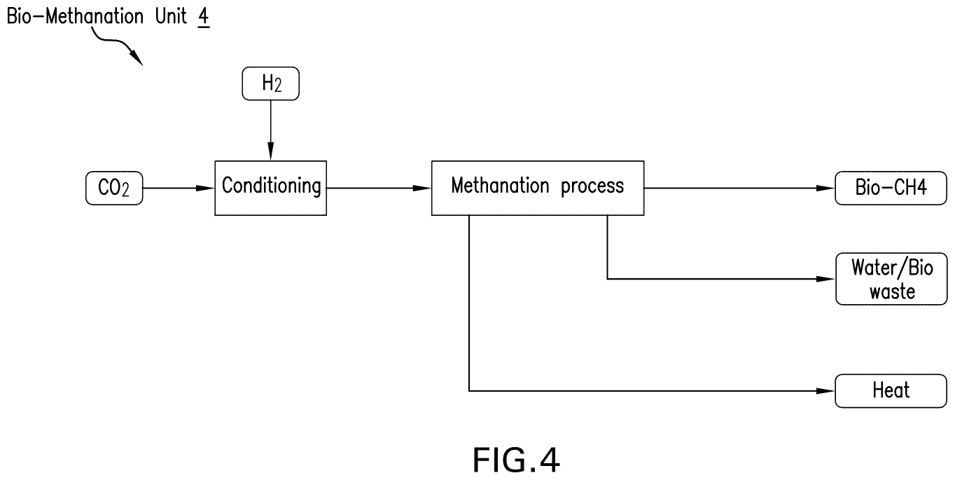

FIG. 4 illustrates a simplified embodiment of the bio-methanation unit 4. In the embodiment of FIG. 4, the bio-methanation unit 4 includes a conditioning unit that receives the separated CO₂ from the CO₂ separation unit 3 and the H₂ gas from the electrolyzer 5. The conditioning unit provides the CO₂ and H₂ gases at a selected pressure and concentration required for the associated bio-methanation process. To do this, the conditioning unit may include (not shown) pressure and concentration sensors, flow control valves, a compressor, and a controller for receiving feedback from the sensors and for controlling the components to achieve the desired pressure and concentration. The bio-methanation unit 4 also includes a methanation reactor (e.g., a reactor vessel) in which micro-organisms synthesize methane from the H₂ gas and the CO₂ received from the conditioning unit. The synthesized methane is then purified to a desired quality in a methane purification unit. Byproducts of the methane synthesis include heat and water. The water may be treated in a waste-water treatment system to a desired quality before being discharged for an intended use. A byproduct of the water treatment process may include bio-waste (e.g. waste biomass/biomass harvested), which optionally may be combusted in the WtE unit 2. Structurally, the bio-methanation unit includes an input port for each of the H₂ gas received from the electrolyzer 5 and the separated CO₂ received from the CO₂ separation unit 3 and an output port for each of the synthesized methane, the water, the bio-waste, and the heat. The methanation reactor among others can be a stirred reactor that may include rotatable impellers to stir methanation components to increase the efficiency of the methanation process. Other types of the methanation reactor may include a bubble column reactor, a loop reactor, or other types of methanation reactors known in the art. In that various bio-methanation units are known in the art, the bio-methanation unit 4 is not discussed in further detail herein.

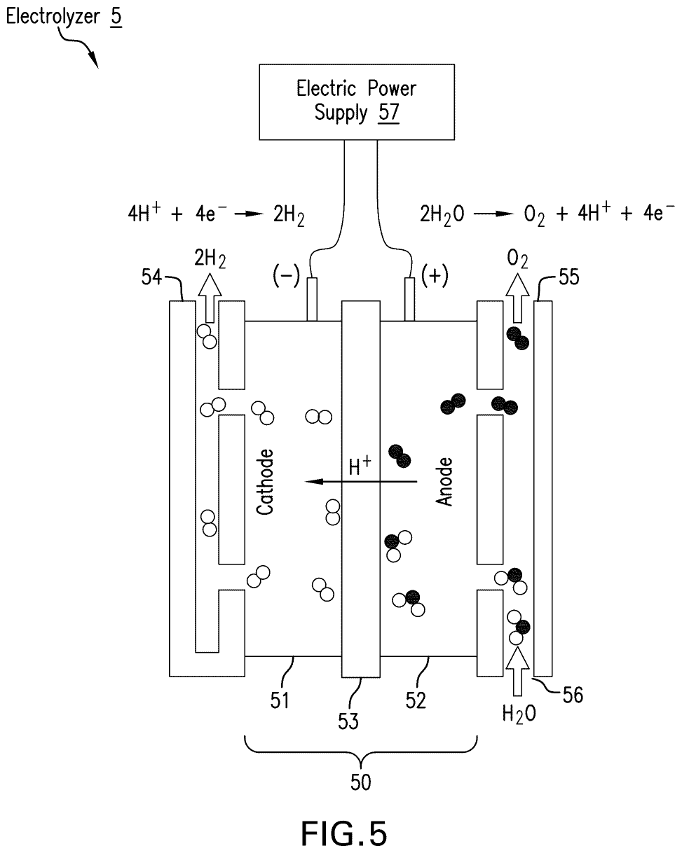

FIG. 5 illustrates a simplified embodiment of the electrolyzer 5. The electrolyzer 5 uses electricity to split water molecules into hydrogen gas and oxygen gas. The electrolyzer 5 includes an electrolytic cell 50 having a cathode 51, which has a negative charge, and an anode 52, which has a positive charge. A membrane or electrolyte 53 separates the cathode 51 from the anode 52. The splitting or electrolysis of the water occurs within the cell when an electric current is applied across the membrane or electrolyte 53. The anode 52 attracts the negatively charged hydroxide ions (OH⁻), releasing oxygen gas (O₂). The cathode 51 attracts the positively charged hydrogen ions (H⁺) and releases hydrogen gas (H₂). The hydrogen gas is released through a hydrogen discharge port 54 and the oxygen gas is released through an oxygen discharge port 55. Water to be split enters the electrolyzer 5 through a water entry port 56. The electrolyzer 5 includes an electric power supply 57 for supplying electricity for the electrolysis. In that various electrolyzers are known in the art, the electrolyzer 5 is not discussed in further detail herein.

FIG. 6 illustrates a first detailed embodiment of the system 10 coupled to the user facility 6. In the embodiment of FIG. 6, the water discharge port of the bio-methanation unit 6 is coupled to the water input of the electrolyzer 5 such that at least some of the water required for the electrolysis is provided by the bio-methanation unit 4. Also, in the embodiment of FIG. 6, the bio-methanation unit 4 is coupled to the user facility 6 to provide methane and heat through associated discharge ports. In one or more embodiments, the heat is provided by a heat transfer fluid in a closed-loop configuration or by mass flow.

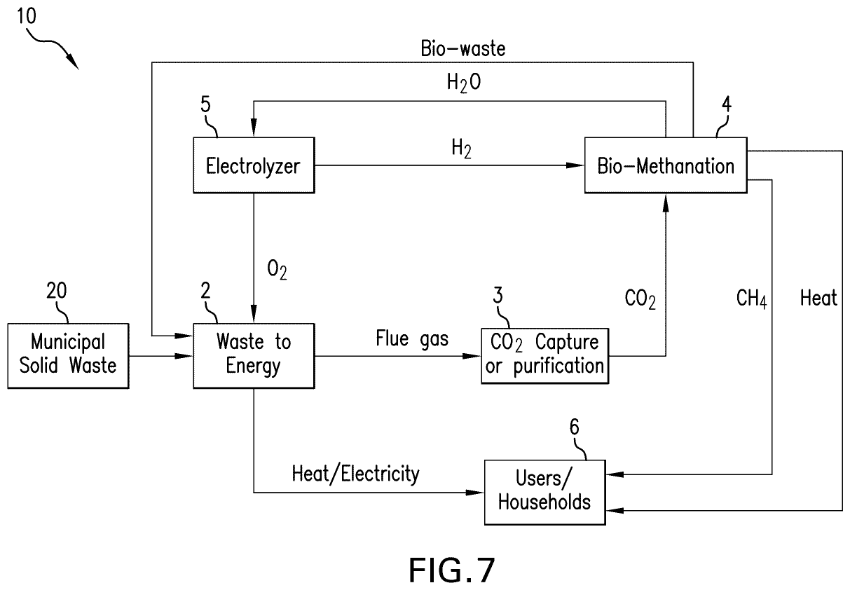

FIG. 7 illustrates a second more detailed embodiment of the system 10 coupled to the user facility 6. In the embodiment of FIG. 7, the bio-methanation unit 4 is configured to produce methane during the bio-methanation process. Byproducts of the bio-methanation process may include wastewater and heat. Wastewater from the bio-methanation may be treated and processed to produce clean water and bio-waste for a final user. The bio-waste obtained from the water treatment is discharged through a bio-waste discharge port that is coupled to a bio-waste input port of the WtE unit 2 where the bio-waste is combusted along with the waste. Also, in the embodiment of FIG. 7, the water discharge port of the bio-methanation unit 4 is coupled to the water input port of the electrolyzer 5 such that at least some of the water required for the electrolysis is provided by the bio-methanation unit 4. Further in the embodiment of FIG. 7, the bio-methanation unit 4 is coupled to the user facility 6 to provide methane and heat through associated discharge ports. In one or more embodiments, the heat is provided by a heat transfer fluid in a closed-loop configuration or by mass flow.

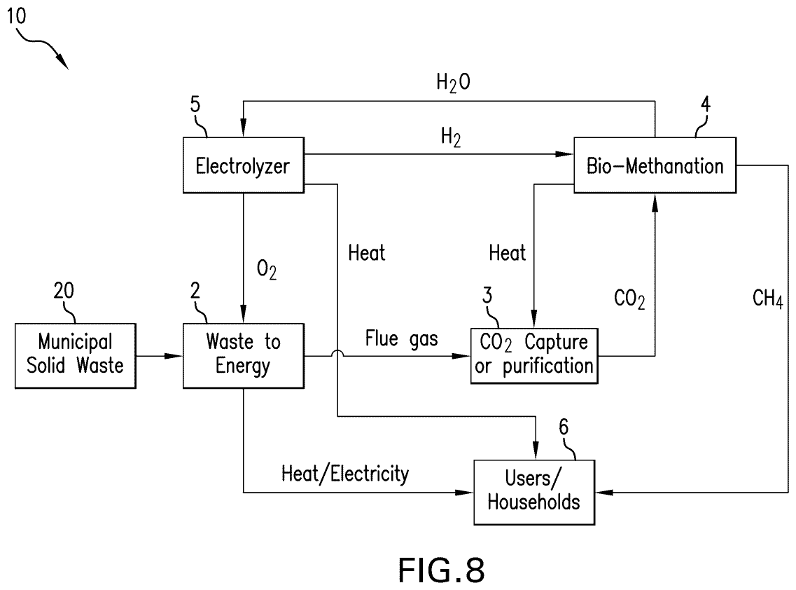

FIG. 8 illustrates a third more detailed embodiment of the system 10 coupled to the user facility 6. In the embodiment of FIG. 8, the electrolyzer 5 is configured to generate heat during the electrolysis process. This heat is discharged through an electrolyzer heat discharge port that is coupled to a heat input port of the user facility 6. Alternatively, or in addition, the heat discharged from the electrolyzer can be provided to the CO₂ separation unit 3 for use in the CO₂ separation process. In one or more embodiments, a coil containing a heat transfer fluid is immersed in the electrolyte and the heat transfer fluid is provided to the user facility 6 and/or to the CO₂ separation unit 3 in a closed-loop configuration. Also, in the embodiment of FIG. 8, the water discharge port of the bio-methanation unit 4 is coupled to the water input port of the electrolyzer 5 such that at least some of the water required for the electrolysis is provided by the bio-methanation unit 4. Further in the embodiment of FIG. 8, the bio-methanation unit 4 is coupled to the user facility 6 to provide methane through the methane discharge port. Further in the embodiment of FIG. 8, the CO₂ separation unit 3 is configured to use heat in the CO₂ separation process. This heat for CO₂ separation is provided by the bio-methanation unit 4 in which the heat discharge port of the bio-methanation unit 4 is coupled to a heat input port of the CO₂ separation unit. The heat may be provided by a heat transfer fluid in a closed-loop configuration.

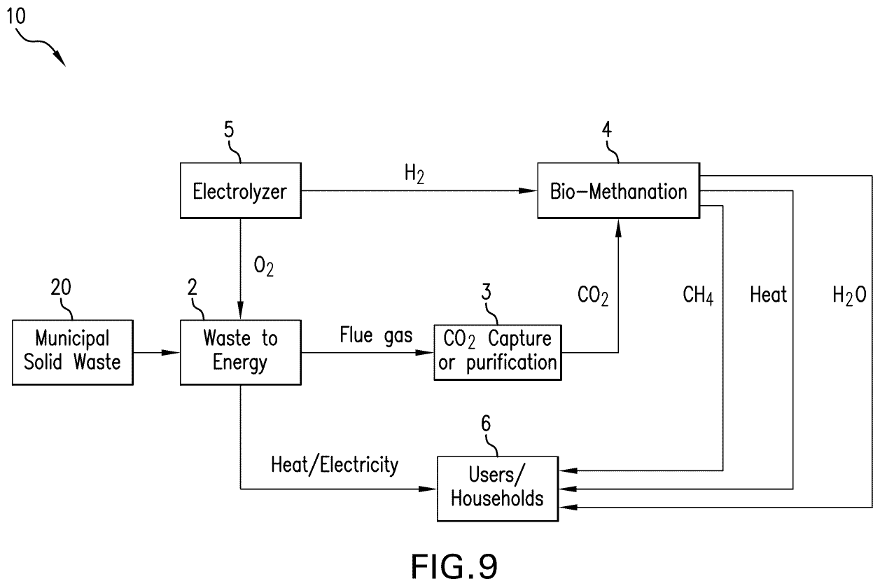

FIG. 9 illustrates a fourth more detailed embodiment of the system 10 coupled to the user facility 6. In the embodiment of FIG. 9, the bio-methanation unit 4 provides methane, heat, and water to the user facility 6. Structurally, the methane output port of the bio-methanation unit 4 is coupled to a methane input port of the user facility 6, the heat output port of the bio-methanation unit 4 is coupled to a heat input port of the user facility 6, and the water output port of the bio-methanation unit 4 is coupled to a water input port of the user facility.

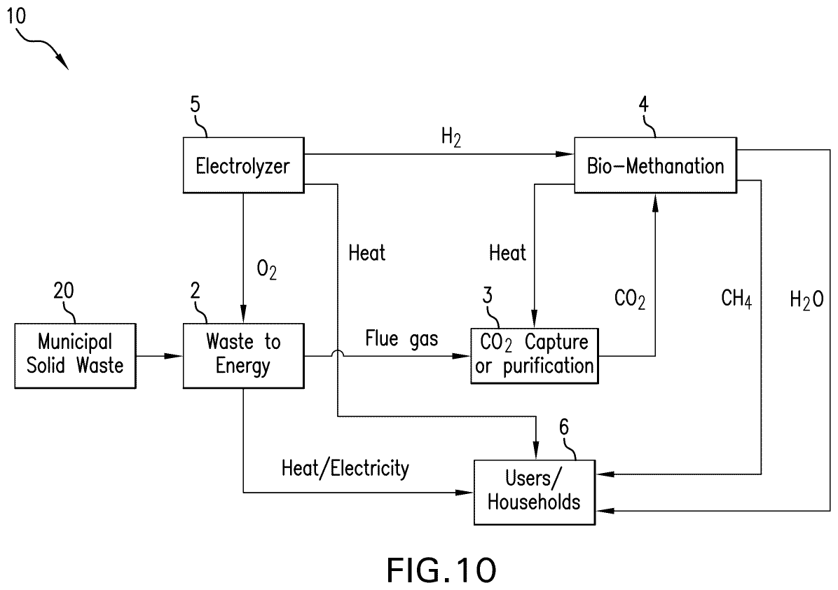

FIG. 10 illustrates a fifth more detailed embodiment of the system 10 coupled to the user facility 6. In the embodiment of FIG. 10, the bio-methanation unit 4 provides methane and water to the user facility 6 and heat to the CO₂ separation unit 3 for the CO₂ separation process. Also, in the embodiment of FIG. 10, the electrolyzer 5 provides heat to the user facility 6. Structurally, the methane output port of the bio-methanation unit 4 is coupled to a methane input port of the user facility 6, the water output port of the bio-methanation unit 4 is coupled to a water input port of the user facility 6, the heat output port of the bio-methanation unit 4 is coupled to a heat input port of the CO₂ separation unit 3, and the heat output port of the electrolyzer 5 is coupled to the heat input port of the user facility 6.

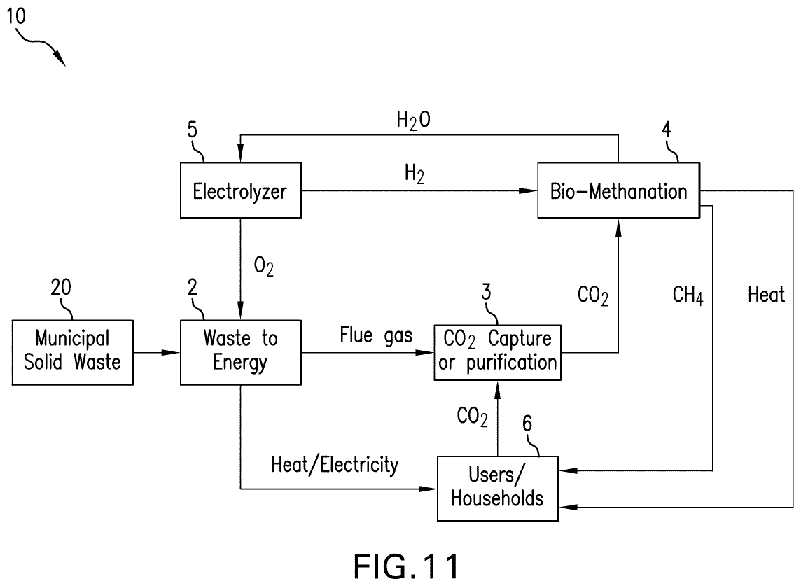

FIG. 11 illustrates a sixth more detailed embodiment of the system 10 coupled to the user facility 6. In the embodiment of FIG. 11, the bio-methanation unit 4 provides methane and heat to the user facility 6 and water to the electrolyzer 5. In addition, the user facility 6 provides CO₂ to the CO₂ separation unit 3. For example, the user facility 6 may be a commercial facility that generates CO₂ from combustion or another commercial process and thus can remediate the production of the CO₂ by sending it to the CO₂ separation unit 3 and then to the bio-methanation unit 4 for the generation of methane. Structurally, the methane output port of the bio-methanation unit 4 is coupled to a methane input port of the user facility 6, the heat output port of the bio-methanation unit 4 is coupled to an associated heat input port of the user facility 6, the water output port of the bio-methanation unit 4 is coupled to a water input port of the electrolyzer 5, and a CO₂ output port of the user facility 6 is coupled to an associated CO₂ input port of the CO₂ separation unit 3.

In one or more embodiments, the electrolyzer 4 generates more oxygen than is needed in the combustion process in the WtE unit 2. In these embodiments, the excess oxygen may be provided to the user facility 6 where the user facility 6 has a use for oxygen. The excess oxygen may be sent via a tubular coupling an excess oxygen output of the of the electrolyzer 4 to an oxygen input of the user facility 6.

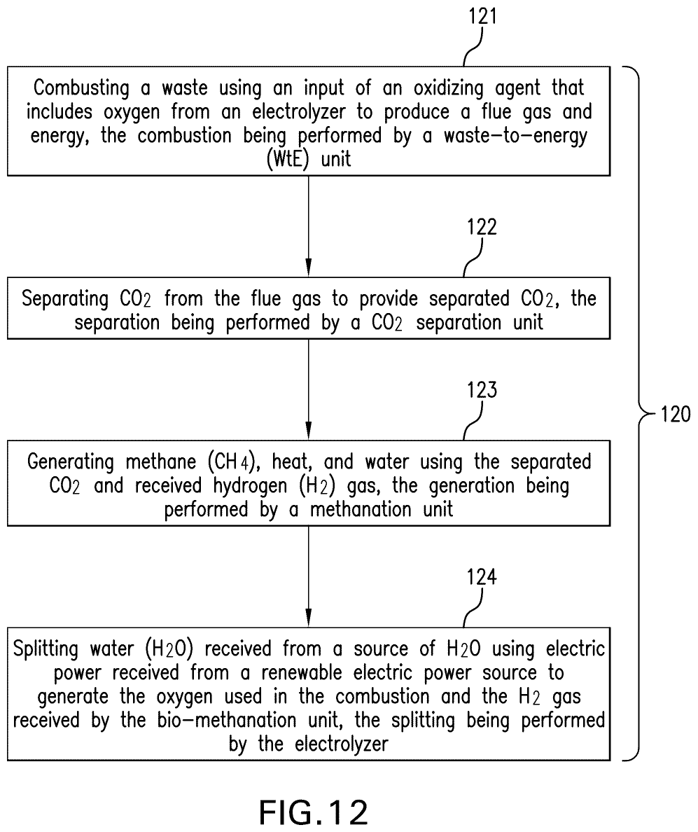

FIG. 12 is a flow chart for a method 120 for producing energy and methane. Block 121 calls for combusting a waste using an input of an oxidizing agent that includes oxygen from an electrolyzer to produce a flue gas and energy, the combustion being performed by a waste-to-energy (WtE) unit. In one or more embodiments, the combusting is performed in a furnace or combustion boiler. Block 121 may also include recirculating the flue gas back through the furnace or combustion boiler or increasing the concentration of O₂ in the oxidizing agent to produce the flue gas with mainly CO₂ and water vapor. Block 122 calls for separating CO₂ from the flue gas to provide separated CO₂, the separation being performed by a CO₂ separation unit. Block 123 calls for generating methane (CH₄), heat, and water using the separated CO₂ and received hydrogen (H₂) gas, the generation being performed by a bio-methanation unit. Block 124 calls for splitting water (H₂O) received from a source of H₂O using electric power received from a renewable electric power source to generate the oxygen used in the combustion and the H₂ gas received by the bio-methanation unit, the splitting being performed by the electrolyzer. In one or more embodiments, the oxidizing agent is air concentrated with oxygen or substantially pure oxygen (e.g., in a range of 95 to 100% oxygen). In one or more embodiments, the electric power source is a green or renewable power source that includes hydropower, wind turbines, or solar cells.

The disclosure herein provides several advantages. With respect to the oxygen-fuel combustion (also referred to as oxy-fuel combustion) by the WtE unit, CO₂ capture is more efficient due to the high concentration of CO₂ and fewer impurities. The WtE unit can provide increased combustion temperature stability and thus increased efficiency of heat transfer. Owing to supply of pure oxygen for the combustion, emission of nitrous oxides (NOₓ), carbon monoxide (CO), and hydrocarbon formation is also reduced. Additionally, oxy-fuel combustion increases convective and radiative heat transfer efficiency. Recirculation of the flue gas increases the efficiency of energy production compared to plants that combust flue gas further downstream.

Advantages with respect to the system include energy recovery from municipal waste to supply user facilities with energy without CO₂ emissions. CO₂ from the WtE unit is captured and utilized with green H₂ gas to produce bio-methane which reduces the CO₂ footprint of municipalities. More services (e.g. heat, power, biomethane) can be provided by the same process entity. Oxygen from the electrolyzer to the WtE unit eliminates the need for an Air-Separation-Unit (ASU) in the WtE unit. Water generated from bio-methanation can be supplied to the electrolyzer and/or user facility. Another advantage is that the utilization of O₂ in WtE unit improves the overall efficiency of the integrated plant. Also, reducing the amount of air in the oxidizer input reduces or eliminates the nitrous oxides (NOx) in the flue gas. Oxy-fuel combustion involves the replacement of air with high purity oxygen, usually above 95 vol %, and recirculated flue gas. Consequently, the produced flue gas stream contains mainly carbon dioxide and water vapor that can be easily removed by condensation.

Embodiment 1: A system for producing energy and methane, the system comprising a waste-to-energy (WtE) unit coupled to a supply of waste and configured to combust the waste using an input of an oxidizing agent having oxygen to produce energy and a flue gas, the WtE unit comprising an oxidizing agent input, a flue gas discharge output, and an energy discharge output; a carbon dioxide (CO₂) separation unit coupled to the flue gas discharge output of the WtE unit and configured to separate CO₂ from the flue gas to provide separated CO₂, the CO₂ separation unit comprising a separated CO₂ discharge output; a bio-methanation unit coupled to the separated CO₂ discharge output of the CO₂ separation unit and configured to generate methane (CH₄), heat, and water using the separated CO₂ received from the CO₂ separation unit and received hydrogen (H₂) gas, the bio-methanation unit comprising a H₂ gas input, a CO₂ gas input, a methane output, a heat output configured, and a water output; and an electrolyzer coupled to a source of water (H₂O) and an electric power source supplying electricity and configured to split the H₂O to generate the oxygen used in the oxidizing agent and the H₂ gas used in the bio-methanation unit, the electrolyzer comprising a water input, an electric power input, a H₂ gas output coupled to the H₂ gas input of the bio-methanation unit, and an oxidizing agent output coupled to the oxidizing agent input of the WtE unit.

Embodiment 2: The system as in any prior embodiment, wherein the oxidizing agent comprises at least one of air with concentrated O₂ or substantially pure oxygen.

Embodiment 3: The system as in any prior embodiment, wherein the energy discharge output of the WtE unit is coupled to an energy input of a user facility and wherein the produced energy comprises at least one of heat and electricity.

Embodiment 4: The system as in any prior embodiment, wherein the water output of the bio-methanation unit is coupled to the water input of the electrolyzer; the methane output of the bio-methanation unit is coupled to a methane input of the user facility; and the heat output of the bio-methanation unit is coupled to a heat input of the user facility.

Embodiment 5: The system as in any prior embodiment, wherein the bio-methanation unit is further configured to generate bio-waste and comprises a bio-waste output, the bio-waste output being coupled to a bio-waste input of the WtE unit that is configured to combust the bio-waste; the methane output of the bio-methanation unit is coupled to a methane input of the user facility; the heat output of the bio-methanation unit is coupled to a bio-methanation heat input of the user facility; and the water output of the bio-methanation unit is coupled to the water input of the electrolyzer.

Embodiment 6: The system as in any prior embodiment, wherein the electrolyzer is further configured to generate heat and further comprises a heat output coupled to another heat input of the user facility; the water output of the bio-methanation unit is coupled to the water input of the electrolyzer; the heat output of the bio-methanation unit is coupled to a heat input of the CO₂ separation unit; and the methane output of the bio-methanation unit is coupled to a methane input of the user facility.

Embodiment 7: The system as in any prior embodiment, wherein the methane output of the bio-methanation unit is coupled to a methane input of the user facility; the heat output of the bio-methanation unit is coupled to a heat input of the user facility; and the water output of the bio-methanation unit is coupled to a water input of the user facility.

Embodiment 8: The system as in any prior embodiment, wherein the electrolyzer is further configured to generate heat and further comprises a heat output coupled to an electrolyzer heat input of the user facility; the heat output of the bio-methanation unit is coupled to a heat input of the CO₂ separation unit; the methane output of the bio-methanation unit is coupled to a methane input of the user facility; and the water output of the bio-methanation unit is coupled to a water input of the user facility.

Embodiment 9: The system as in any prior embodiment, wherein the water output of the bio-methanation unit is coupled to the water input of the electrolyzer; the methane output of the bio-methanation unit is coupled to a methane input of the user facility; the heat output of the bio-methanation unit is coupled to a heat input of the user facility; and the user facility produces CO₂ and comprises a CO₂ output coupled to a user CO₂ input of the CO₂ separation unit.

Embodiment 10: The system as in any prior embodiment, wherein the supply of waste is a municipal solid waste supply.

Embodiment 11: The system as in any prior embodiment, wherein the WtE unit comprises a combustion boiler that is configured to heat a working fluid by the combusting of the solid waste to produce the working fluid in a high energy state; an expander coupled to the combustion boiler and configured to receive the working fluid in the high energy state and to convert energy of the working fluid to mechanical energy by expansion of the working fluid in the high energy state; an electric generator coupled to a mechanical output of the expander and configured to generate electricity; and an energy exchanger coupled to a working fluid output of the expander and configured to extract heat energy from the working fluid downstream of the expander.

Embodiment 12: A method for producing energy and methane, the method including combusting a waste using an input of an oxidizing agent that includes oxygen from an electrolyzer to produce a flue gas and energy, the combustion being performed by a waste-to-energy (WtE) unit; separating CO₂ from the flue gas to provide separated CO₂, the separation being performed by a CO₂ separation unit; generating methane (CH₄), heat, and water using the separated CO₂ and received hydrogen (H₂) gas, the generation being performed by a bio-methanation unit; and splitting water (H₂O) received from a source of H₂O using electric power received from a renewable electric power source to generate the oxygen used in the combustion and the H₂ gas received by the bio-methanation unit, the splitting being performed by the electrolyzer.

Embodiment 13: The method as in any prior embodiment, wherein the oxidizing agent comprises at least one of air, air with concentrated O₂, and substantially pure oxygen.

Embodiment 14: The method as in any prior embodiment, further comprising sending the energy produced by the WtE unit to a user facility, the energy comprising at least one of heat or generated electricity.

Embodiment 15: The method as in any prior embodiment, further comprising sending the water generated by the bio-methanation unit to the electrolyzer; sending the methane generated by the bio-methanation unit to the user facility; and sending the heat generated by the bio-methanation unit to the user facility.

Embodiment 16: The method as in any prior embodiment, further comprising generating bio-waste with the bio-methanation unit and sending the bio-waste to the WtE unit that combusts the bio-waste; sending the methane generated by the bio-methanation unit to the user facility; sending the heat generated by the bio-methanation unit to the user facility; and sending the water generated by the bio-methanation unit to the electrolyzer.

Embodiment 17: The method as in any prior embodiment, further comprising generating heat using the electrolyzer and sending the heat to the user facility; sending water generated by the bio-methanation unit to the electrolyzer; sending the heat generated by the bio-methanation unit to the CO₂ separation unit; and sending the methane generated by the bio-methanation unit to the user facility.

Embodiment 18: The method as in any prior embodiment, further comprising sending the methane generated by the bio-methanation unit to the user facility; sending the heat generated by the bio-methanation unit to the user facility; and sending the water generated by the bio-methanation unit to the user facility.

Embodiment 19: The method as in any prior embodiment, further comprising generating heat using the electrolyzer and sending the heat to the user facility; sending the heat generated by the bio-methanation unit to the CO₂ separation unit; sending the methane generated by the bio-methanation unit to the user facility; and sending the water generated by the bio-methanation unit to the user facility.

Embodiment 20: The method as in any prior embodiment, further comprising sending water generated by the bio-methanation unit to the electrolyzer; sending the methane generated by the bio-methanation unit to the user facility; sending the heat generated by the bio-methanation unit to the user facility; and sending CO₂ produced by the user facility to the CO₂ separation unit.

Embodiment 21: The method as in any prior embodiment, wherein the electrolyzer generates excess oxygen that is more than is needed for the combustion and the method further comprises sending the excess oxygen to the user facility.

Embodiment 22: The method as in any prior embodiment, wherein the combusting comprises combusting the waste in a combustion boiler that generates the flue gas and heats a working fluid to produce the working fluid in a high energy state; expanding the working fluid in the high energy state using an expander to convert energy of the working fluid to mechanical energy; generating electricity using an electric generator that receives the mechanical energy; extracting energy from the working fluid downstream of the expander using an energy exchanger; and sending at least one of the extracted energy from the heat exchanger or the electricity to the user facility.

Elements of the embodiments have been introduced with either the articles “a” or “an.” The articles are intended to mean that there are one or more of the elements. The terms “including” and “having” and the like are intended to be inclusive such that there may be additional elements other than the elements listed. The conjunction “or” when used with a list of at least two terms is intended to mean any term or combination of terms. The term “configured” relates to one or more structural limitations of a device that are required for the device to perform the function or operation for which the device is configured. The limitations may be known in the art for a specific item, but not known in the context of or application to the invention as a whole. The limitations may be inclusive of circuit modules and software known to perform a specific function. The term “coupled” relates to being coupled directly or indirectly using an intermediate device. The terms “first” and “second” and like are used to distinguish terms and not to denote a particular order.

The flow diagram depicted herein is just an example. There may be many variations to this diagram or the steps (or operations) described therein without departing from the scope of the invention. For example, operations may be performed in another order or other operations may be performed at certain points without changing the specific disclosed sequence of operations with respect to each other. All of these variations are considered a part of the claimed invention.

The disclosure illustratively disclosed herein may be practiced in the absence of any element which is not specifically disclosed herein.

While one or more embodiments have been shown and described, modifications and substitutions may be made thereto without departing from the scope of the invention. Accordingly, it is to be understood that the present invention has been described by way of illustrations and not limitation.

It will be recognized that the various components or technologies may provide certain necessary or beneficial functionality or features. Accordingly, these functions and features as may be needed in support of the appended claims and variations thereof, are recognized as being inherently included as a part of the teachings herein and a part of the invention disclosed.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed:

1. A system for producing energy and methane, the system comprising:
a waste-to-energy (WtE) unit coupled to a supply of waste and configured to combust the waste using an input of an oxidizing agent having oxygen to produce energy and a flue gas, the WtE unit comprising an oxidizing agent input, a flue gas discharge output, and an energy discharge output;
a carbon dioxide (CO₂) separation unit coupled to the flue gas discharge output of the WtE unit and configured to separate CO₂ from the flue gas to provide separated CO₂, the CO₂ separation unit comprising a separated CO₂ discharge output;
a bio-methanation unit coupled to the separated CO₂ discharge output of the CO₂ separation unit and configured to generate methane (CH₄), heat, and water using the separated CO₂ received from the CO₂ separation unit and received hydrogen (H₂) gas, the bio-methanation unit comprising a H₂ gas input, a CO₂ gas input, a methane output, a heat output configured, and a water output; and
an electrolyzer coupled to a source of water (H₂O) and an electric power source supplying electricity and configured to split the H₂O to generate the oxygen used in the oxidizing agent and the H₂ gas used in the bio-methanation unit, the electrolyzer comprising a water input, an electric power input, a H₂ gas output coupled to the H₂ gas input of the bio-methanation unit, and an oxidizing agent output coupled to the oxidizing agent input of the WtE unit.

2. The system according to claim 1, wherein the oxidizing agent comprises at least one of air with concentrated O₂ or substantially pure oxygen.

3. The system according to claim 1, wherein the energy discharge output of the WtE unit is coupled to an energy input of a user facility and wherein the produced energy comprises at least one of heat and electricity.

4. The system according to claim 3, wherein: the water output of the bio-methanation unit is coupled to the water input of the electrolyzer; the methane output of the bio-methanation unit is coupled to a methane input of the user facility; and the heat output of the bio-methanation unit is coupled to a heat input of the user facility.

5. The system according to claim 3, wherein: the bio-methanation unit is further configured to generate bio-waste and comprises a bio-waste output, the bio-waste output being coupled to a bio-waste input of the WtE unit that is configured to combust the bio-waste; the methane output of the bio-methanation unit is coupled to a methane input of the user facility; the heat output of the bio-methanation unit is coupled to a bio-methanation heat input of the user facility; and the water output of the bio-methanation unit is coupled to the water input of the electrolyzer.

6. The system according to claim 3, wherein: the electrolyzer is further configured to generate heat and further comprises a heat output coupled to another heat input of the user facility; the water output of the bio-methanation unit is coupled to the water input of the electrolyzer; the heat output of the bio-methanation unit is coupled to a heat input of the CO₂ separation unit; and the methane output of the bio-methanation unit is coupled to a methane input of the user facility.

7. The system according to claim 3, wherein: the methane output of the bio-methanation unit is coupled to a methane input of the user facility; the heat output of the bio-methanation unit is coupled to a heat input of the user facility; and the water output of the bio-methanation unit is coupled to a water input of the user facility.

8. The system according to claim 3, wherein: the electrolyzer is further configured to generate heat and further comprises a heat output coupled to an electrolyzer heat input of the user facility; the heat output of the bio-methanation unit is coupled to a heat input of the CO₂ separation unit; the methane output of the bio-methanation unit is coupled to a methane input of the user facility; and the water output of the bio-methanation unit is coupled to a water input of the user facility.

9. The system according to claim 3, wherein: the water output of the bio-methanation unit is coupled to the water input of the electrolyzer; the methane output of the bio-methanation unit is coupled to a methane input of the user facility; the heat output of the bio-methanation unit is coupled to a heat input of the user facility; and the user facility produces CO₂ and comprises a CO₂ output coupled to a user CO₂ input of the CO₂ separation unit.

10. The system according to claim 1, wherein the supply of waste is a municipal solid waste supply.

11. The system according to claim 1, wherein the WtE unit comprises:
a combustion boiler that is configured to heat a working fluid by the combusting of the solid waste to produce the working fluid in a high energy state;
an expander coupled to the combustion boiler and configured to receive the working fluid in the high energy state and to convert energy of the working fluid to mechanical energy by expansion of the working fluid in the high energy state;
an electric generator coupled to a mechanical output of the expander and configured to generate electricity; and
an energy exchanger coupled to a working fluid output of the expander and configured to extract heat energy from the working fluid downstream of the expander.

12. A method for producing energy and methane, the method comprising:
combusting a waste using an input of an oxidizing agent that includes oxygen from an electrolyzer to produce a flue gas and energy, the combustion being performed by a waste-to-energy (WtE) unit;
separating CO₂ from the flue gas to provide separated CO₂, the separation being performed by a CO₂ separation unit;
generating methane (CH₄), heat, and water using the separated CO₂ and received hydrogen (H₂) gas, the generation being performed by a bio-methanation unit; and
splitting water (H₂O) received from a source of H₂O using electric power received from a renewable electric power source to generate the oxygen used in the combustion and the H₂ gas received by the bio-methanation unit, the splitting being performed by the electrolyzer.

13. The method according to claim 12, wherein the oxidizing agent comprises at least one of air, air with concentrated O₂, and substantially pure oxygen.

14. The method according to claim 12, further comprising sending the energy produced by the WtE unit to a user facility, the energy comprising at least one of heat or generated electricity.

15. The method according to claim 14, further comprising: sending the water generated by the bio-methanation unit to the electrolyzer; sending the methane generated by the bio-methanation unit to the user facility; and sending the heat generated by the bio-methanation unit to the user facility.

16. The method according to claim 14, further comprising: generating bio-waste with the bio-methanation unit and sending the bio-waste to the WtE unit that combusts the bio-waste; sending the methane generated by the bio-methanation unit to the user facility; sending the heat generated by the bio-methanation unit to the user facility; and sending the water generated by the bio-methanation unit to the electrolyzer.

17. The method according to claim 14, further comprising: generating heat using the electrolyzer and sending the heat to the user facility; sending water generated by the bio-methanation unit to the electrolyzer; sending the heat generated by the bio-methanation unit to the CO₂ separation unit; and sending the methane generated by the bio-methanation unit to the user facility.

18. The method according to claim 14, further comprising: sending the methane generated by the bio-methanation unit to the user facility; sending the heat generated by the bio-methanation unit to the user facility; and sending the water generated by the bio-methanation unit to the user facility.

19. The method according to claim 14, further comprising: generating heat using the electrolyzer and sending the heat to the user facility; sending the heat generated by the bio-methanation unit to the CO₂ separation unit; sending the methane generated by the bio-methanation unit to the user facility; and sending the water generated by the bio-methanation unit to the user facility.

20. The method according to claim 14, further comprising: sending water generated by the bio-methanation unit to the electrolyzer; sending the methane generated by the bio-methanation unit to the user facility; sending the heat generated by the bio-methanation unit to the user facility; and sending CO₂ produced by the user facility to the CO₂ separation unit.

21. The method according to claim 14, wherein the electrolyzer generates excess oxygen that is more than is needed for the combustion and the method further comprises sending the excess oxygen to the user facility.

22. The method according to claim 14, wherein the combusting comprises:
combusting the waste in a combustion boiler that generates the flue gas and heats a working fluid to produce the working fluid in a high energy state;
expanding the working fluid in the high energy state using an expander to convert energy of the working fluid to mechanical energy;
generating electricity using an electric generator that receives the mechanical energy;
extracting energy from the working fluid downstream of the expander using an energy exchanger; and sending at least one of the extracted energy from the heat exchanger or the electricity to the user facility.
